(19) United States
(12) Patent Application Publication
(10) Pub. No.: US 2026/0201302 A1
(43) Pub. Date: Jul. 16, 2026

(54) LOW ENERGY CONSUMPTION FERMENTATION DEVICE, LOW ENERGY CONSUMPTION FERMENTATION SYSTEM AND FERMENTATION METHOD

(71) Applicant: CHANT OIL CO., LTD., New Taipei City (TW)

(72) Inventors: Ming-Jui WANG, New Taipei City (TW); Li-Hsueh CHANG, New Taipei City (TW); Chia-Chi LIN, New Taipei City (TW); Ting-Chun KUO, New Taipei City (TW); Po-Hsun CHANG, New Taipei City (TW); Shun-Niang CHEN, New Taipei City (TW); Yung-Che LU, New Taipei City (TW); Wen-Jie CHANG, New Taipei City (TW); Guan-Chiun LEE, New Taipei City (TW)

(21) Appl. No.: 19/313,845

(22) Filed: Aug. 28, 2025

(30) Foreign Application Priority Data
Jan. 15, 2025 (TW) 114101694
Apr. 17, 2025 (TW) 114203842

(51) Int. Cl. C12M 1/06; C12M 1/00; C12M 1/34; C12M 1/36

(52) U.S. Cl. CPC C12M 27/02; C12M 29/06; C12M 41/12; C12M 41/26; C12M 41/32; C12M 41/48

(57) ABSTRACT

The present disclosure provides a low energy consumption fermentation system and a fermentation method. The low energy consumption fermentation system includes a fermentation tank, a feeding tank, an aeration device, a stirring shaft, a fermentation broth feeding tank, at least one adding tank and an automatic control device. The feeding tank is coupled to the fermentation tank. The aeration device is disposed at a bottom of the fermentation tank. The stirring shaft is installed upright in the fermentation tank, and a plurality of stirring blades is disposed on the stirring shaft. The fermentation broth feeding tank is coupled to the fermentation tank. The addition tank is coupled to the fermentation tank. The automatic control device is coupled to the feeding tank, the aeration device, the stirring shaft, the fermentation broth feeding tank, and at least one adding tank.

### CROSS-REFERENCE TO RELATED APPLICATION

This application claims priority to Taiwan Application Serial Number 114101694, filed Jan. 15, 2025, and Taiwan Application Serial Number 114203842, filed Apr. 17, 2025, all of which are herein incorporated by reference.

### BACKGROUND

### Field of Invention

The present disclosure relates to a low energy consumption fermentation device, a low energy consumption fermentation system and a low energy fermentation method.

### Description of Related Art

The microorganism used in aerobic fermentation technology is a unicellular fungus, which can be yeast or other aerobic fermentation bacteria. It is easy to grow and harmless, and is often used in large quantities in the brewing production industry. Yeast can decompose macromolecules into small molecules that are easy for cell metabolism and utilization, and can also be used in related research such as cell culture and genetic engineering. The related biological culture system has developed rapidly in recent years and has been widely used in various fields and has been favored.

With the increase in various products, fermentation byproducts and market demand including microbial cells and other related biomass, the fermentation process of the existing fermentation tank has been improved and applied. The fermentation tank uses aerobic fermentation as its main process. The aeration equipment is mainly installed at the bottom of the fermentation tank or in the space below the fermentation tank to allow oxygen or mixed gases containing other oxygen to enter the fermentation tank from the aeration equipment, thereby enabling yeast or other microorganisms in the fermentation tank to perform aerobic respiration of cells. The microbubbles created by delivering oxygen through aeration equipment can disturb the fluid and enhance uniformity in the fermentation tank.

Aerobic fermentation production currently still requires manual addition of culture medium into the fermentation tank at regular intervals. Without real-time monitoring of the manufacturing process, it is easy for unnecessary large molecular proteins or other microbial bodies to form in the fermentation tank, causing contamination.

### SUMMARY

The present disclosure provides a low energy consumption fermentation system, which including a fermentation tank, a feeding tank, an aeration device, a stirring shaft, a fermentation broth feeding tank, at least one adding tank, and an automatic control device. The feeding tank is coupled to the fermentation tank. The aeration device is disposed at a bottom of the fermentation tank. The stirring shaft is installed upright in the fermentation tank, in which a plurality of stirring blades is disposed on the stirring shaft. The fermentation broth feeding tank is coupled to the fermentation tank. The at least one adding tank is coupled to the fermentation tank. The automatic control device is coupled to the feeding tank, the aeration device, the stirring shaft, the fermentation broth feeding tank, and the at least one adding tank.

In some embodiments, the low energy consumption fermentation system further includes at least one sensor disposed on the fermentation tank and the at least one sensor coupled to the automatic control device.

In some embodiments, the low energy consumption fermentation system further includes an air flow meter coupled to the aeration device.

In some embodiments, the stirring blades on the stirring shaft are used to maintain a tip speed of 0.3 m/s to 5.8 m/s.

In some embodiments, the stirring blades include three pairs of the stirring blades spaced apart and disposed on the stirring shaft, each pair of the stirring blades is symmetrically disposed on the stirring shaft, adjacent pair of the stirring blades are spaced apart by a distance, and a ratio of the distance to a diameter of the fermentation tank is 1/4 to 1/5.

In some embodiments, the fermentation tank has a liquid level and a ratio of the distance to the liquid level is 1/3 to 1/6.

In some embodiments, the aeration device includes a plurality of pores and a diameter of each of the pores is 0.5 micrometers to 10 micrometers.

The present disclosure further provides a low energy fermentation method, which includes the following steps. An aerobic microorganism is fed into the fermentation tank through the feed tank of the low energy fermentation system as mentioned above, so that the aerobic microorganism is mixed with a culture medium fluid in the fermentation tank. A sterile gas flows into the fermentation tank through the aeration device. The aerobic microorganism, the sterile gas and the culture medium fluid are stirred in the fermentation tank by the stirring shaft. A tip speed of each stirring blades on the stirring shaft is maintain at 0.3 m/s to 5.8 m/s.

In some embodiments, the sterile gas flows into the fermentation tank through the aeration device at a flow rate of 0.5 L/min to 5 L/min.

In some embodiments, flowing a sterile gas into the fermentation tank through the aeration device includes maintaining a dissolved oxygen value in the fermentation tank at 35% to 45% by the automatic control device.

The present disclosure further provides a low energy consumption fermentation device. The low energy consumption fermentation device includes a fermentation tank, a feeding tank, a fermentation broth feeding tank, a sensor, an aeration device. The feeding tank is communicated with the fermentation tank. The fermentation broth feeding tank is communicated with the fermentation tank. The sensor is disposed on the fermentation tank, in which an angle between the sensor and a sidewall of the fermentation tank is 45 degrees to 75 degrees. The aeration device is disposed below the fermentation tank and communicated with a bottom of the fermentation tank.

In some embodiments, the low energy consumption fermentation device further includes an automatic control device electrically connected to the feeding tank, the fermentation broth feeding tank, the sensor, and the aeration device.

In some embodiments, the low energy consumption fermentation device further includes an adding tank communicated with the fermentation tank and electrically connected to the automatic control device.

In some embodiments, the adding tank includes a defoamer adding tank, an acid solution adding tank, an alkaline solution adding tank, or a combination therefore.

In some embodiments, the sensor includes a dissolved oxygen sensor, a temperature sensor, a pH sensor, or a combination therefore.

In some embodiments, the low energy consumption fermentation device further includes an air flow meter coupled to the aeration device.

In some embodiments, the aeration device includes a plurality of pores and a diameter of each of the pores is 0.5 micrometers to 10 micrometers.

In some embodiments, the low energy consumption fermentation device further includes a stirring shaft installed upright in the fermentation tank, of which a plurality of stirring blades is disposed on the stirring shaft.

In some embodiments, the stirring blades include three pairs of the stirring blades spaced apart and disposed on the stirring shaft, each pair of the stirring blades is symmetrically disposed on the stirring shaft, adjacent pair of the stirring blades are spaced apart by a distance, and a ratio of the distance to a diameter of the fermentation tank is 1/4 to 1/5.

In some embodiments, the fermentation tank has a liquid level and a ratio of the distance to the liquid level is 1/3 to 1/6.

It is to be understood that the foregoing general description and the following detailed description are merely exemplary and explanatory, and are intended to provide further illustration of the present disclosure.

### BRIEF DESCRIPTION OF THE DRAWINGS

The present disclosure can be more fully understood by reading the following detailed description of the embodiments, with reference made to the accompanying drawings.

FIG. 1 is a schematic diagram of a low energy fermentation system according to various embodiments of the present disclosure.

FIG. 2 is a schematic diagram of a low energy fermentation device according to various embodiments of the present disclosure.

FIG. 3 is a flowchart of a low energy fermentation method according to various embodiments of the present disclosure.

### DETAILED DESCRIPTION

Reference will now be made in detail to the embodiments of the present disclosure, examples of which are illustrated in the accompanying drawings. Wherever possible, the same reference numbers are used in the drawings and the description to refer to the same or like parts.

The following embodiments are disclosed with accompanying diagrams for detailed description. For illustration clarity, many details of practice are explained in the following descriptions. However, it should be understood that these details of practice do not intend to limit the present disclosure. That is, these details of practice are not necessary in parts of embodiments of the present disclosure. Furthermore, for simplifying the drawings, some of the conventional structures and elements are shown with schematic illustrations.

The term “about,” “approximately,” or “substantially” as used herein is inclusive of the stated value and means within an acceptable range of deviation for the particular value as determined by persons of ordinary skill in the art, considering the measurement in question and the error associated with measurement of the particular quantity (i.e., the limitations of the measurement system). For example, “about” may mean within one or more standard deviations, or within, for example, +30%, +20%, +15%, +10%, +5% of the stated value. Moreover, a relatively acceptable range of deviation or standard deviation may be chosen for the term “about,” “approximately,” or “substantially” as used herein based on measuring properties, coating properties or other properties, instead of applying one standard deviation across all the properties.

Although below using a series of actions or steps described in this method disclosed, but the order of these actions or steps shown should not be construed to limit the present invention. For example, certain actions or steps may be performed in different orders and/or concurrently with other steps. Moreover, not all steps must be performed in order to achieve the depicted embodiment of the present invention. Furthermore, each operation or procedure described herein may contain several sub-steps or actions.

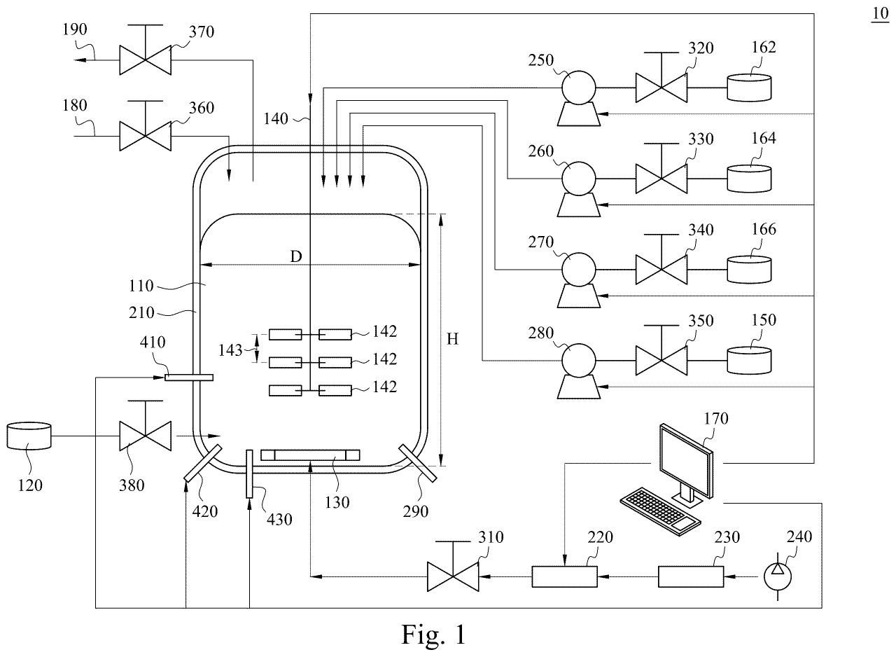

The present disclosure provides a low energy consumption fermentation system for performing a continuous large-scale aerobic fermentation production process for producing aerobic microorganisms. This system can avoid the unnecessary macromolecular proteins or contamination caused by other microorganisms, and compared with traditional fermentation systems, the low energy consumption fermentation system disclosed herein can save at least 65% of energy. FIG. 1 is a schematic diagram of a low energy fermentation system according to various embodiments of the present disclosure. Please refer to FIG. 1, the low energy consumption fermentation system 10 includes a fermentation tank 110, a feeding tank 120, an aeration device 130, a stirring shaft 140, a fermentation broth feeding tank 150, at least one adding tank 162, 164, 166, and an automatic control device 170. To be specific, the feeding tank 120 is coupled to the fermentation tank 110. The aeration device 130 is disposed at a bottom of the fermentation tank 110. The stirring shaft 140 is installed upright in the fermentation tank 110, and a plurality of stirring blades 142 is disposed on the stirring shaft 140. The fermentation broth feeding tank 150 is coupled to the fermentation tank 110. The at least one adding tank 162, 164, 166 is coupled to the fermentation tank 110. The automatic control device 170 is coupled to the feeding tank 120, the aeration device 130, the stirring shaft 140, the fermentation broth feeding tank 150, and the at least one adding tank 162, 164, 166. In some embodiments, the automatic control device 170 includes an artificial intelligence (AI).

In some embodiments, the fermentation tank 110 has an inner space. The fermentation tank 110 is a vertical cylindrical structure with a capacity of 5 liters to 1000 liters and a diameter D. In some embodiments, the low energy consumption fermentation system 10 further includes a temperature control component 210 covering the fermentation tank 110 for controlling the temperature of the fermentation tank 110.

The feeding tank 120 is used to transport aerobic microorganisms to the fermentation tank 110. In some embodiments, a valve 380 may be provided between the feeding tank 120 and the fermentation tank 110 to control the amount of aerobic microorganisms entering the fermentation tank 110.

In some embodiments, the low energy consumption fermentation system 10 further includes an air flow meter 220, a gas filter 230, and a gas compressor 240. To be specific, the air flow meter 220 is coupled to the aeration device 130, the gas filter 230 is coupled to the air flow meter 220, and the gas compressor 240 is coupled to the gas filter 230. The air flow meter 220 is used to monitor the change of gas flow direction and to continuously measure the flow of materials within a certain period of time. The purpose of providing the gas filter 230 is to filter bacteria in the gas and remove unwanted or unnecessary excess gas to obtain sterile gas. In some embodiments, a valve 310 may be disposed between the air flow meter 220 and the aeration device 130 to close or open the gas, change the flow rate, control the flow direction, adjust the downstream pressure, and adjust the pressure when the system pressure exceeds or falls below a predetermined range.

In some embodiments, the stirring blades 142 on the stirring shaft 140 include at least one pair of stirring blades 142, and the at least one pair of stirring blades 142 are symmetrically disposed in the stirring shaft 140. In some embodiments, the stirring blades 142 on the stirring shaft 140 include three pairs of stirring blades 142 spaced apart and disposed on the stirring shaft 140, each pair of stirring blades 142 is symmetrically disposed on the stirring shaft 140, the adjacent pair of stirring blades 142 are spaced apart by a distance 143, and a ratio of the distance 143 to the diameter D of the fermentation tank 110 is 1/4 to 1/5, as shown in FIG. 1. The number of stirring blades 142 on the stirring shaft 140 may be designed according to the volume of the fermentation tank 110, but the present disclosure is not limited thereto. The stirring shaft 140 and the plurality of stirring blades 142 thereon are disposed above the aeration device 130. The stirring blades 142 stir the fluid in the fermentation tank 110 to generate a vortex. The vortex may drive the microbubbles generated by the aeration device 130 to be evenly distributed in the mixed fluid of the aerobic microorganisms and the culture medium fluid in the fermentation tank 110. The stirring shaft 140 and the plurality of stirring blades 142 thereon in combination with the aeration device 130 not only enhance the uniformity and the transmission quality, but also further increase the dissolved oxygen content in the fermentation tank 110.

It is noted that when the low energy consumption fermentation system 10 is on operation, the stirring blades 142 on the stirring shaft 140 are used to maintain a tip speed of 0.3 m/s to 5.8 m/s. It should be noted that the “tip speed” referred to here is the speed of the tip (the end point farthest from the stirring shaft 140) of each stirring blade 142 when it rotates. When the tip speed of each stirring blade 142 is maintained within the above range, the power consumption of the low energy consumption fermentation system 10 can be greatly reduced.

The relationship between the distance 143 between adjacent pairs of stirring blades 142 and the height of the fluid in the fermentation tank 110 affects the flow pattern and mixed forms. In some embodiments, the fermentation tank 110 has a liquid level H and a ratio of the distance 143 between adjacent pairs of stirring blades 142 to the liquid level His 1/3 to 1/6. In one embodiment, when the volume of the fermentation tank 110 is within the range of minimum (5 L) and maximum (500 L), in order to maintain good axial flow and sufficient mixing, the ratio of the distance 143 between the adjacent pairs of stirring blades 142 to the liquid level H is preferably 1/4.

In some embodiments, a valve 350 and a pump 280 may be disposed between the fermentation broth feeding tank 150 and the fermentation tank 110 to continuously supply nutrients consumed by aerobic microorganisms to the fermentation tank 110, so that nutrients can be automatically supplied to the fermentation tank 110 under predetermined time conditions, thereby promoting the growth and yield of aerobic microorganisms and realizing the automation of continuous feeding.

For example, the fermentation broth feeding tank 150 includes YPG culture medium, YAG culture medium, or a combination thereof. To be specific, the YPG culture medium includes yeast extract, peptone and glycerol, and the molar ratio of the three is 1:1:1 to 1:2:2. Specifically, the YAG culture medium includes yeast extract, ammonium sulfate and glycerol, and the molar ratio of the three is 1:1:1 to 1:2:30.

In some embodiments, the number of adding tanks coupled to the fermentation tank 110 may be plural. For example, the adding tanks include a defoamer adding tank 162, an acid solution adding tank 164, and an alkaline solution adding tank 166. These adding tanks are independently coupled to the fermentation tank 110.

Furthermore, a valve 320 and a pump 250 may be disposed between the defoamer adding tank 162 and the fermentation tank 110 to suppress the foam generated by the mixed liquid in the fermentation tank 110.

Similarly, a valve 330 and a pump 260 may be disposed between the acid solution adding tank 164 and the fermentation tank 110 to control the pH value of the mixed liquid in the fermentation tank 110.

Similarly, a valve 340 and a pump 270 may be disposed between the alkaline solution adding tank 166 and the fermentation tank 110 to control the pH value of the mixed liquid in the fermentation tank 110.

In some embodiments, the low energy consumption fermentation system 10 may further include at least one sensor disposed on the fermentation tank 110, and the at least one sensor is coupled to the automatic control device 170. In some embodiments, the number of sensors may be plural. For example, the sensors include a dissolved oxygen sensor 410, a temperature sensor 420, and a pH sensor 430. These sensors are used to detect whether the mixed solution in the fermentation tank 110 is maintained within a predetermined range.

As shown in FIG. 1, the automatic control device 170 is coupled to the feeding tank 120, the aeration device 130, the stirring shaft 140, the fermentation broth feeding tank 150, the defoamer adding tank 162, the acid solution adding tank 164, and the alkaline solution adding tank 166. More specifically, the automatic control device 170 is electrically connected to the pumps 250, 260, 270, and 280, the air flow meter 220, the stirring shaft 140, the dissolved oxygen sensor 410, the temperature sensor 420, and the pH sensor 430.

With reference to FIG. 1, the low energy consumption fermentation system 10 may further include a sterilization steam pipeline 180 and an exhaust pipeline 190, each of which is disposed at the top of the fermentation tank 110. In some embodiments, a valve 360 may be disposed on the sterilization steam pipeline 180 to control whether high-temperature steam can enter the fermentation tank 110 from the sterilization steam pipe 180. In some embodiments, a valve 370 may be disposed on the exhaust pipeline 190 to control whether the gas can be discharged from the exhaust pipe 190 and to control the pressure in the fermentation tank 110 at the same time. In some embodiments, the low energy consumption fermentation system 10 further includes a discharge opening 290 for discharging the mixed solution of microorganisms and culture medium fluid.

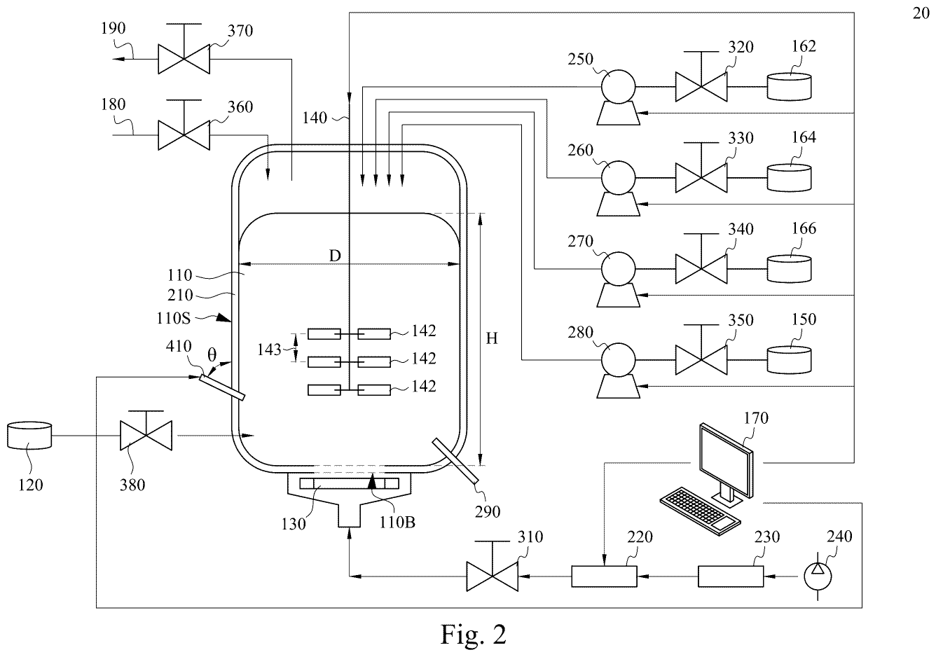

The present disclosure also provides a low energy consumption fermentation device to further enhance the sensitivity of the sensors. FIG. 2 is a schematic diagram of a low energy fermentation device according to various embodiments of the present disclosure. Please refer to FIG. 2. The low energy consumption fermentation device 10 includes the fermentation tank 110, the feeding tank 120, the fermentation broth feeding tank 150, a sensor 410, and the aeration device 130. Specifically, the feeding tank 120 is communicated with the fermentation tank 110. The fermentation broth feeding tank 150 communicated with the fermentation tank 110. The sensor 410 is disposed on the fermentation tank 110, of which an angle θ between the sensor 410 and a sidewall 110S of the fermentation tank 110 is 45 degrees to 75 degrees. The aeration device 130 is disposed below the fermentation tank 110 and communicated with a bottom 110B of the fermentation tank 110. In order to make it easy to compare the differences between the above-mentioned embodiments and the embodiments in FIG. 2 and simplify the descriptions, the same symbols are used to label the same members in the following various embodiments and mainly the differences between the various embodiments are described while repetitive parts are not described again.

It is worth noting that the various sensors 410 are all disposed obliquely on the sidewall 110S of the fermentation tank 110 as shown in FIG. 2. In other words, the angle θ between each sensor 410 and the sidewall 110S of the fermentation tank 110 is 45 degrees to 75 degrees, for example, 50 degrees, 55 degrees, 60 degrees, 65 degrees, or 70 degrees. This design may further improve the sensitivity of the sensors 410. Specifically, when the gas enters the fermentation tank 110 from the aeration device 130 below, the gas is guided to diffuse upward along the tilt angle of the sensors 410 without causing the gas to gather at the bottoms of the sensors 410, thereby reducing the error of the detection value. Especially in the later stage of the aerobic fermentation process, as the viscosity of the liquid in the fermentation tank 110 increases, it is more difficult for the gas to diffuse evenly. This design can better prevent the gas from being blocked at the bottoms of the sensors 410.

It is worth noting that, as shown in FIG. 2, since the aeration device 130 is disposed below the fermentation tank 110, it is convenient in that the aeration device 130 can be easily removed from the fermentation tank 110 and a cleaning process of the aeration device 130 can be performed. In other words, the aeration device 130 is a detachable aeration device 130. For example, the aeration device 130 and the bottom 110B of the fermentation tank 110 may be connected and/or separated from each other by means of slide rails, buckles, hinges, screws, or a combination thereof.

In some embodiments, the aeration device 130 includes a ventilation base (not shown) and an aeration plate structure (not shown) above the aeration plate, in which the aeration plate structure includes an air chamber formed by a protective ring (not shown), a multi-layer sintered stacked hollow structure (not shown) and a membrane with a plurality of pores (not shown). For example, the aeration plate structure is evenly distributed with a plurality of pores, and the average pore size (diameter) of each pore may be 0.5 μm to 10 μm. The aeration plate structure with micropores of appropriate size can be selected based on the gas flow rate. Appropriate pore size and distance distribution can prevent microbubbles from quickly agglomerating to form larger bubbles, thereby allowing the gas to be evenly distributed in the mixed fluid of aerobic microorganisms and culture medium fluid in the form of microbubbles. Furthermore, the aeration plate structure can provide microbubbles with a size of about 0.5 μm to about 10 μm, so that the mixed fluid in the fermentation tank 110 can be fully in contact with the gas. In the above-mentioned pore size range, the size of microbubbles can be improved by adjusting the total flow rate and pressure of the introduced gas. Microbubble formation can increase the solubility of oxygen. Microbubbles of appropriate size can evenly distribute the gas in the mixed fluid of the aerobic microorganisms and the culture medium fluid to increase the dissolved oxygen content of the mixed fluid and enhance the uniformity in the fermentation tank 110.

Furthermore, the dissolved oxygen (DO) in the fermentation tank 110 has an important influence on the growth of microorganisms and product formation. During the fermentation process, an appropriate amount of sterile air must be supplied so that the microorganisms can reproduce and accumulate the required metabolites. Because during the fermentation process, as the microorganisms rapidly grow and enter the logarithmic growth phase, the oxygen consumption of the microorganisms increases significantly. At this time, if the gas supply is terminated, the oxygen in the mixed liquid in the fermentation tank 110 will be quickly consumed by the microorganisms. Generally speaking, oxygen does not dissolve easily in water. At atmospheric pressure and 25° C., the concentration of oxygen dissolved in water is approximately 0.25 mg/L. Under the same conditions, the low energy consumption fermentation system 10 disclosed herein can significantly increase the concentration of oxygen dissolved in water to 15 mg/L.

The low energy consumption fermentation device of the present disclosure may further enhance the sensitivity of the sensors by installing the sensors obliquely to the sidewall of the fermentation tank. In addition, the low energy consumption fermentation device of the present disclosure is provided by placing the aeration device below the fermentation tank, so that the aeration device can be easily disassembled for subsequent cleaning.

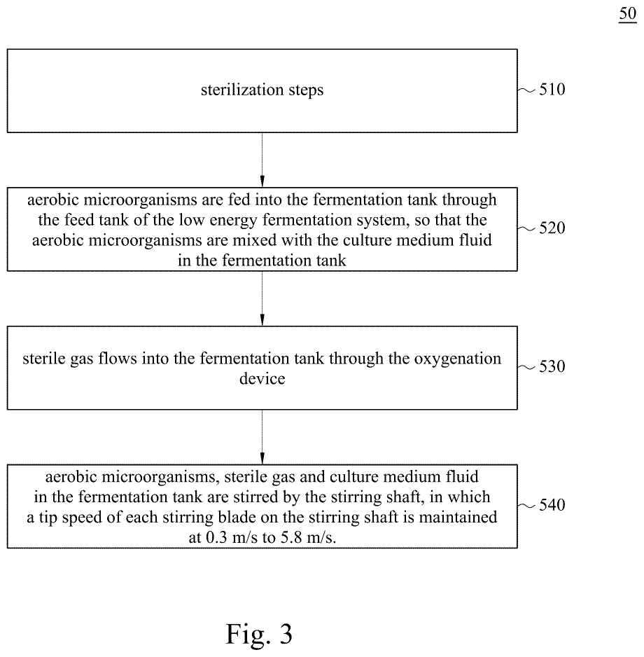

The present disclosure further provides a low energy fermentation method for performing a continuous large-scale aerobic fermentation production process for producing aerobic microorganisms. This method can not only reduce the energy consumption of the aforementioned fermentation system, but also reduce production costs. FIG. 3 is a flowchart of a low energy fermentation method according to various embodiments of the present disclosure. The low energy fermentation method 50 includes at least step 510, step 520, step 530, and step 540. Step 510 is a sterilization step. Specifically, please refer to FIG. 1 and FIG. 3 at the same time. The sterilization step includes introducing high temperature steam into the fermentation tank 110 for 30 to 60 minutes, wherein the temperature of the high temperature steam is 110° C. to 130° C. More specifically, under atmospheric pressure, the valve 360 is opened, and high temperature steam with a temperature of 110° C. to 130° C. is continuously introduced from the sterilization steam pipeline 180 into the fermentation tank 110 for sterilization. After the sterilization is completed, the valve 370 is opened, and the sterilized high temperature steam is discharged from the fermentation tank 110 through the exhaust pipeline 190.

In step 520, aerobic microorganisms are transported to the fermentation tank 110 through the feeding tank 120 of the low energy consumption fermentation system 10, so that the aerobic microorganisms are mixed with the culture medium fluid in the fermentation tank.

In step 530, the sterile gas flows into the fermentation tank 110 through the oxygenation device 130. Specifically, the gas compressor 240 is turned on, and the gas (e.g., air) is filtered through the gas filter 230 to obtain sterile gas, and then the automatic control device 170 controls the flow rate of the air flow meter 220 to continuously deliver the sterile gas to the fermentation tank 110. The sterile gas enters the air holes on the aeration device 130 at the bottom of the fermentation tank 110 and generates tiny and dense microbubbles in the fermentation tank 110. The microbubbles are evenly distributed in the mixed solution of aerobic microorganisms and the culture medium fluid to improve the uniformity in the fermentation tank 110.

In some embodiments, the flow rate of the sterile gas transmitted to the fermentation tank 110 through the aeration device 130 is 0.5 L/min to 5 L/min. The flow rate of the introduced gas can be adjusted according to demand, but the present disclosure is not limited thereto. It should be noted that in this system, the operations of the gas compressor 240 and the stirring shaft 140 are the main energy consumption source. Therefore, when the required gas flow rate in the fermentation tank 110 is higher, more energy is consumed. On the contrary, when the flow rate of the gas used in the fermentation tank 110 is lower, energy is saved more.

In some embodiments, the aforementioned sterile gas includes oxygen, and the content of oxygen accounts for 15% to 25% of the content of the sterile gas.

In step 540, the aerobic microorganisms, the sterile gas and the culture medium fluid in the fermentation tank 110 are stirred by the stirring shaft 140, wherein a tip speed of each stirring blade 142 on the stirring shaft 140 is maintained at 0.3 m/s to 5.8 m/s. Specifically, the tip speed of the stirring blades 142 on the stirring shaft 140 is preset to fall within the above range by the automatic control device 170, and then the stirring shaft 140 stirs the fermentation tank 110 to generate a vortex. The vortex can drive the microbubbles generated by the aeration device 130 to be evenly distributed in the mixed solution in the fermentation tank 110, which not only improves the uniformity and transmission quality, but also increases the dissolved oxygen content in the fermentation tank 110.

In some embodiments, the dissolved oxygen value in the fermentation tank 110 is maintained at 35% to 45% by the automatic control device 170. For example, when the dissolved oxygen sensor 410 electrically connected to the automatic control device 170 detects that the dissolved oxygen content in the fermentation tank 110 is not within the expected range, the automatic control device 170 will control the air flow meter 220 to continue to introduce sterile gas into the fermentation tank 110, thereby realizing the automation of numerical control of dissolved oxygen in the fermentation tank 110.

In some embodiments, the pH value of the fermentation tank 110 is maintained at 4.5 to 6.0 by the automatic control device 170. For example, when the pH sensor 430 electrically connected to the automatic control device 170 detects that the pH value in the fermentation tank 110 is not within the expected range, the automatic control device 170 will control the pump 260 and/or 270 to automatically add acidic or alkaline solution into the fermentation tank 110, thereby realizing the automation of numerical control of pH in the fermentation tank 110.

In some embodiments, when the automatic control device 170 detects that foam is generated violently in the fermentation tank 110, the automatic control device 170 will control the pump 250 to automatically add a defoaming agent into the fermentation tank 110 to suppress the foam in the fermentation tank 110, thereby preventing excessive foam from affecting and possibly contaminating the mixed solution of aerobic microorganisms and culture medium fluid in the fermentation tank 110.

In some embodiments, step 540 further includes maintaining the temperature of the fermentation tank 110 at 20° C. to 40° C. This temperature range is the optimal growth temperature for aerobic microorganisms.

The following provides some detailed embodiments to help those with ordinary knowledge in the technical field better understand the present disclosure. The provision of detailed embodiments is not intended to limit the scope of the present disclosure to the description of the embodiments.

In order to compare the power consumption of the traditional fermentation system and the low energy consumption fermentation system of the present disclosure, the power consumption data of Embodiments 1-3 and Comparative Examples 1-3 in the present experimental example will be collected and counted.

In this experimental example, Comparative Examples 1-3 are based on the conventional fermentation system, wherein the stirring speed of which is controlled to be 3.665 m/s (equivalent to 1000 rpm), and aerobic microorganisms are continuously produced for 120 hours. Embodiments 1-3 are based on the low energy consumption fermentation system of the present disclosure, wherein the stirring speed is controlled to be 1.099 m/s (equivalent to 300 rpm), and aerobic microorganisms are continuously produced for 120 hours. The production hours and power consumption of Comparative Examples 1-3 and Embodiments 1-3 are shown in Table 1 below.

TABLE 1

| | Production hours | Power consumption |
|---|---|---|
| | (hours) | (Watts) |
| Comparative Example 1 | 120 | 6179 |
| Comparative Example 2 | 120 | 6495 |
| Comparative Example 3 | 120 | 6521 |
| Embodiment 1 | 120 | 848 |
| Embodiment 2 | 120 | 890 |
| Embodiment 3 | 120 | 885 |

It can be seen from Table 1 above that the electricity consumed by the traditional fermentation system is at least 7 times the electricity consumed by the low energy consumption fermentation system of the present disclosure. In other words, using the low energy consumption fermentation system of the present disclosure can save at least 7 times of energy.

In some embodiments, the dry cell weight (DCW) of yeast obtained by the low energy fermentation method 50 is not much different from that obtained by the traditional high-speed stirring fermentation method. The final dry weight of the culture obtained by both methods is 95 g±5 g.

Although the present disclosure has been described in considerable detail with reference to certain embodiments, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the present disclosure. In view of the foregoing, it is intended that the present disclosure cover the modifications and variations of the present disclosure falling within the scope of the appended claims.

What is claimed is:

1. A low energy consumption fermentation system, comprising:
a fermentation tank;
a feeding tank coupled to the fermentation tank;
an aeration device disposed at a bottom of the fermentation tank;
a stirring shaft installed upright in the fermentation tank, wherein a plurality of stirring blades is disposed on the stirring shaft;
a fermentation broth feeding tank coupled to the fermentation tank;
at least one adding tank coupled to the fermentation tank; and
an automatic control device coupled to the feeding tank, the aeration device, the stirring shaft, the fermentation broth feeding tank, and the at least one adding tank.

2. The low energy consumption fermentation system of claim 1, further comprising at least one sensor disposed on the fermentation tank and the at least one sensor coupled to the automatic control device.

3. The low energy consumption fermentation system of claim 1, further comprising an air flow meter coupled to the aeration device.

4. The low energy consumption fermentation system of claim 1, wherein the stirring blades on the stirring shaft are used to maintain a tip speed of 0.3 m/s to 5.8 m/s.

5. The low energy consumption fermentation system of claim 1, wherein the stirring blades comprise three pairs of the stirring blades spaced apart and disposed on the stirring shaft, each pair of the stirring blades is symmetrically disposed on the stirring shaft, adjacent pair of the stirring blades are spaced apart by a distance, and a ratio of the distance to a diameter of the fermentation tank is 1/4 to 1/5.

6. The low energy consumption fermentation system of claim 5, wherein the fermentation tank has a liquid level and a ratio of the distance to the liquid level is 1/3 to 1/6.

7. The low energy consumption fermentation system of claim 1, wherein the aeration device comprises a plurality of pores and a diameter of each of the pores is 0.5 micrometers to 10 micrometers.

8. A low energy fermentation method, the method comprising:
feeding an aerobic microorganism into the fermentation tank through the feed tank of the low energy consumption fermentation system of claim 1, so that the aerobic microorganism is mixed with a culture medium fluid in the fermentation tank;
flowing a sterile gas into the fermentation tank through the aeration device; and
stirring the aerobic microorganism, the sterile gas and the culture medium fluid in the fermentation tank by the stirring shaft, wherein a tip speed of each stirring blades on the stirring shaft is maintain at 0.3 m/s to 5.8 m/s.

9. The method of claim 8, wherein the sterile gas flows into the fermentation tank through the aeration device at a flow rate of 0.5 L/min to 5 L/min.

10. The method of claim 8, wherein flowing a sterile gas into the fermentation tank through the aeration device comprises maintaining a dissolved oxygen value in the fermentation tank at 35% to 45% by the automatic control device.

11. A low energy consumption fermentation device, comprising:
a fermentation tank;
a feeding tank communicated with the fermentation tank;
a fermentation broth feeding tank communicated with the fermentation tank;
a sensor disposed on the fermentation tank, wherein an angle between the sensor and a sidewall of the fermentation tank is 45 degrees to 75 degrees; and
an aeration device disposed below the fermentation tank and communicated with a bottom of the fermentation tank.

12. The low energy consumption fermentation device of claim 11, further comprising an automatic control device electrically connected to the feeding tank, the fermentation broth feeding tank, the sensor, and the aeration device.

13. The low energy consumption fermentation device of claim 12, further comprising an adding tank communicated with the fermentation tank and electrically connected to the automatic control device.

14. The low energy consumption fermentation device of claim 13, wherein the adding tank comprises a defoamer adding tank, an acid solution adding tank, an alkaline solution adding tank, or a combination therefore.

15. The low energy consumption fermentation device of claim 11, wherein the sensor comprises a dissolved oxygen sensor, a temperature sensor, a pH sensor, or a combination therefore.

16. The low energy consumption fermentation device of claim 11, further comprising an air flow meter coupled to the aeration device.

17. The low energy consumption fermentation device of claim 11, wherein the aeration device comprises a plurality of pores and a diameter of each of the pores is 0.5 micrometers to 10 micrometers.

18. The low energy consumption fermentation device of claim 11, further comprising a stirring shaft installed upright in the fermentation tank, wherein a plurality of stirring blades is disposed on the stirring shaft.

19. The low energy consumption fermentation device of claim 18, wherein the stirring blades comprise three pairs of the stirring blades spaced apart and disposed on the stirring shaft, each pair of the stirring blades is symmetrically disposed on the stirring shaft, adjacent pair of the stirring blades are spaced apart by a distance, and a ratio of the distance to a diameter of the fermentation tank is 1/4 to 1/5.

20. The low energy consumption fermentation device of claim 19, wherein the fermentation tank has a liquid level and a ratio of the distance to the liquid level is 1/3 to 1/6.
